(19) United States
(12) Patent Application Publication
(10) Pub. No.: US 2024/0058526 A1
(43) Pub. Date: Feb. 22, 2024

(54) ADHESIVE LAYER WITH MULTIPLE STRENGTHS OF ADHESIVES FOR SECURING AN ON-BODY MEDICAL DEVICE TO A USER

(71) Applicant: INSULET CORPORATION, Acton, MA (US)

(72) Inventors: Kyle BREINGAN, Lowell, MA (US); Alexander DOUDOUMOPOULOS, Boxborough, MA (US)

(21) Appl. No.: 18/450,081

(22) Filed: Aug. 15, 2023

Related U.S. Application Data
(60) Provisional application No. 63/371,776, filed on Aug. 18, 2022.

(51) Int. Cl. A61M 5/142

(52) U.S. Cl. CPC A61M 5/14248; A61M 2025/0266

(57) ABSTRACT

In exemplary embodiments, an on-body medical device has an adhesive layer with adhesives of two or more strengths. In some of the exemplary embodiments, a stronger adhesive is positioned on the adhesive layer where additional strength is needed, such as around the perimeter of the adhesive layer to prevent premature peeling. A weaker adhesive is positioned on other portions of the adhesive layer where there is no need for the strength of the stronger adhesive. In other exemplary embodiments, three strengths of adhesives are used on the adhesive layer. An intermediate strength adhesive is used along with a stronger adhesive and a weaker adhesive. The intermediate strength adhesive may be used in areas where the weaker adhesive does not provide enough strength but the full strength of the stronger adhesive is not needed.

### RELATED APPLICATIONS

This application claims the benefit of U.S. Provisional Patent Application No. 63/371,776, filed Aug. 18, 2022, the entire contents of which are incorporated herein by reference in their entirety.

### BACKGROUND

On-body medical devices often include adhesive layers for securing the on-body medical devices to the user. The adhesive layers contain adhesives that adhere to the skin of the users to hold the on-body medical devices in place on the users. To secure such an on-body medical device to the skin, the user presses the adhesive layer against the skin until the adhesive layer securely adheres to the skin. Most on-body medical devices have fairly short lifespans of use, such as for a few hours or a few days. When the end of the lifespan of use is reached, the on-body medical device is removed by peeling the adhesive layer off of the skin of the user.

Choosing the best adhesive for an adhesive layer of an on-body medical device can be difficult. The adhesive must be strong enough to keep the on-body medical device secured to the user for the entire lifespan of use. Otherwise, the adhesive may fail and the on-body medical device may become detached from the user. However, the adhesive also must not be too strong. If the adhesive is too strong, it may be painful for the user to peel off the adhesive layer at the end of the lifespan of use. A too strong adhesive also may cause skin damage to the user when the adhesive layer is peeled off. Most conventional on-body medical devices tend to have strong adhesives to prevent detachment of the on-body medical devices from the skins of the users.

### SUMMARY

In accordance with an inventive facet, an on-body medical device includes a skin-facing surface configured for facing the skin of a user. The device also includes an adhesive layer secured to the skin-facing surface for securing the on-body medical device to the skin of the user. The adhesive layer includes a stronger adhesive positioned around an outer perimeter of the adhesive layer and a weaker adhesive positioned inside the outer perimeter of the adhesive layer. The stronger adhesive is at least 20% stronger than the weaker adhesive.

The on-body medical device may additionally include a needle and/or cannula insertion assembly for inserting a needle and/or cannula through the skin of the user to deliver a medicament to the user. The device may also include an opening in the skin-facing surface, and an opening in the adhesive layer that is aligned with the opening in the skin facing surface to create a path for the needle and/or cannula to pass for enabling the needle and/or the cannula to be inserted through the skin of the user. The stronger adhesive also may surround a perimeter of the opening in the adhesive layer. The device may include an intermediate strength adhesive that surrounds the opening in the adhesive layer. The intermediate strength adhesive may be weaker than the stronger adhesive but stronger than the weaker adhesive. The weaker adhesive may be a 1 day wear time adhesive or a 2 day wear time adhesive. The stronger adhesive may be a 3 day wear time adhesive or a 4 day wear time adhesive. In this case, “wear time” may refer to an average wear time for a typical user under typical conditions. Alternatively, the stronger adhesive may be 10% to 100% stronger than the weaker adhesive, or more particularly, 10% to 50%; or in an exemplary embodiment, 20% stronger. The on-body medical device may be a medicament delivery device. The medicament delivery device may be an insulin pump. The skin-facing surface may be a surface on the housing of the on-body medical device. The device may include a hole in the adhesive layer, and the stronger adhesive may be positioned on the adhesive layer around a perimeter of the hole.

In accordance with another inventive facet, an on-body medical device includes a housing having a skin-facing surface configured for facing the skin of a user. The device also includes an adhesive layer secured to the skin-facing surface of the housing for securing the on-body medical device to the skin of the user. The adhesive layer includes what may be termed “islands” on the adhesive layer where no adhesive is present or where a weaker strength adhesive (relative to the other adhesive) is present, and may include a stronger strength adhesive applied around a perimeter of the adhesive layer, and may also include a stronger strength adhesive around a region interior to the perimeter so as to surround the islands. Accordingly, the islands of no adhesive or weaker strength may be defined by their complete perimeter being surrounded by a stronger adhesive. The stronger strength adhesive may be 10% to 100% stronger than the weaker adhesive, or more particularly, 10% to 50%; or in an exemplary embodiment, at least 20% stronger than the weaker strength adhesive.

The islands may be periodically spaced apart. The islands may be arranged in a grid. The islands may have the weaker adhesive present, and the weaker adhesive may be a 1 day wear time or a 2 day wear time adhesive. The stronger adhesive may be a 3 day wear time or a 4 day wear time adhesive. In some embodiments, the stronger adhesive may be a 3 to 28 days wear time adhesive, more specifically a 3 to 10 days wear time adhesive and in particular a 3 to 5 days wear time adhesive. A 1 day wear time adhesive may be defined as an adhesive that looses more than 50% of its peel strength within 1 day of being in contact with the user's skin. A 3 day wear time adhesive may be defined as an adhesive that loses more than 50% of its peel strength within 3 day of being in contact with the user's skin, but less than 50% within 2 days of being in contact with the user's skin. In some embodiments, the stronger adhesive is an acrylate based adhesive. In some embodiments, the weaker adhesive is a silicone based adhesive.

In accordance with an additional inventive facet, an on-body medical device includes a skin-facing surface configured for facing the skin of a user and an adhesive layer secured to the skin-facing surface for securing the on-body medical device to the skin of the user. In reverse of the earlier embodiment, the adhesive layer may include “islands” on the adhesive layer where a stronger strength adhesive is present and a weaker strength adhesive applied around a perimeter of the adhesive layer and also applied around a region interior to the perimeter so as to surround the islands with the weaker strength adhesive. Accordingly, the islands of stronger adhesive may be defined by their complete perimeter being surrounded by a weaker adhesive. The stronger strength adhesive may be 10% to 100% stronger than the weaker adhesive, or more particularly, 10% to 50%; or in an exemplary embodiment, at least 20% stronger than the weaker strength adhesive.

The islands may be periodically spaced apart. The islands may be arranged in a grid. The device may further include a needle and/or cannula insertion assembly for inserting a needle and/or cannula through the skin of the user to deliver a medicament to the user, an opening in the skin-facing surface, and a corresponding opening in the adhesive layer for enabling the needle and/or the cannula to be inserted through the skin of the user.

In another exemplary embodiment, the adhesive may form a gradient of strength from an outer perimeter (where the adhesive may be stronger) toward the center (where the adhesive may be weaker). The stronger adhesive at the perimeter may be 10% to 100% stronger than the weaker adhesive, or more particularly, 10% to 50%; or in an exemplary embodiment, 20% stronger. And moving in from the perimeter, the adhesive may gradually weaken until it becomes what may be termed the “weaker adhesive.” This gradient may be created by applying different amounts of adhesive across the surface of the adhesive layer, such that the perimeter has a greater amount of adhesive material than the interior or near the center of the adhesive layer, which may result in a stronger adhesive property.

In a further exemplary embodiment, a portion of the perimeter may have a weaker adhesive then other portions of the perimeter or than the rest of the perimeter. For example, a bottom end of the adhesive layer may have a weaker adhesive that is easier to separate from a user's skin, so as to allow the user to insert their finger at that portion under the adhesive layer and above the skin when it comes time to remove the adhesive layer from the skin. This portion may be arcuate in shape or in the shape of a fingertip. By allowing a user to insert their fingertip at this weaker adhesive portion when it comes time to remove the adhesive layer from the skin, it may be easier for the user to start the adhesive removal process of the rest of the adhesive layer.

### BRIEF DESCRIPTION OF THE DRAWINGS

FIG. 1A depicts a side view of an illustrative on-body medical device of an exemplary embodiment that includes an adhesive pad.

FIG. 1B depicts a partially exploded view of the adhesive pad and the housing of the on-body medical device of FIG. 1A.

FIG. 2 depicts a flowchart of illustrative steps that may be performed in exemplary embodiments to create and secure an adhesive layer to an on-body medical device.

FIG. 3 depicts an illustrative bottom surface of an on-body medical device of an exemplary embodiment having at least two adhesives of different strengths on an adhesive layer.

FIG. 4 depicts a flowchart of illustrative steps that may be performed in exemplary embodiments to create an adhesive layer having at least two adhesives of different strengths.

FIG. 5 depicts an illustrative bottom surface of an on-body medical device of an exemplary embodiment having adhesives of at least three strengths on an adhesive layer.

FIG. 6 depicts a flowchart of illustrative steps that may be performed in exemplary embodiments to create an adhesive layer having at least three adhesives of different strengths.

FIG. 7 depicts an illustrative bottom surface of an on-body medical device of an exemplary embodiment having “islands” of adhesive or lack of adhesive and a surrounding region.

FIG. 8 depicts a flowchart of illustrative steps that may be performed in exemplary embodiments to apply a stronger adhesive to islands and a weaker adhesive to surrounding spaces on an adhesive layer of an on-body medical device.

FIG. 9A depicts an illustrative bottom surface of an on-body medical device of an exemplary embodiment where holes and other structures may have a stronger adhesive applied around the holes or structures to prevent peeling.

FIG. 9B depicts an illustrative bottom surface of an on-body medical device of an exemplary embodiment where adhesive forms a gradient of lessening strength as one moves radially inward from a stronger adhesive at the perimeter to a weaker adhesive at the center of the on-body medical device.

FIG. 9C depicts an illustrative bottom surface of an on-body medical device of an exemplary embodiment where a portion of the perimeter has a weaker adhesive then other portions of the perimeter or than the rest of the perimeter to allow the user to insert their finger at that portion under the adhesive layer.

### DETAILED DESCRIPTION

In exemplary embodiments, an on-body medical device has an adhesive layer with adhesives of two or more strengths. In some of the exemplary embodiments, a stronger adhesive is positioned on the adhesive layer where additional strength is needed, such as around the perimeter of the adhesive layer to prevent premature peeling. A weaker adhesive is positioned on other portions of the adhesive layer where there is no need for the strength of the stronger adhesive. For example, the interior portion of the adhesive layer inside the perimeter may be coated with the weaker adhesive. The use of the weaker adhesive in the interior may reduce the discomfort to a user when peeling off the adhesive layer and may decrease the risk of skin damage. The stronger adhesive around the perimeter may still securely hold the adhesive layer to the skin of the user but may occupy only a thin band around the perimeter. As a result, it may not be as painful to peel off the adhesive layer of exemplary embodiments as it is to peel off a conventional adhesive layer with a strong adhesive coated on the entire adhesive layer since the band of stronger adhesive used in the exemplary embodiments only occupies a small surface area.

In other exemplary embodiments, at least three strengths of adhesives are used on the adhesive layer. An intermediate strength adhesive is used along with a stronger adhesive and a weaker adhesive. The stronger adhesive and weaker adhesive may be positioned largely as discussed above, but the intermediate strength adhesive may be used in areas where the weaker adhesive does not provide enough strength but the full strength of the stronger adhesive is not needed.

In some exemplary embodiments, the adhesives may be applied continuously over designated regions that cover large portions of the surface area of the adhesive layer. In other exemplary embodiments, one or more of the adhesives may be applied in small discrete regions, such as dots or islands, which may be surrounded by other adhesives on the adhesive layer. The discrete adhesive regions may be organized into a pattern, such as a grid. The discrete regions may provide discrete points or islands of stronger adhesive or discrete points or islands of weaker strength adhesive in exemplary embodiments.

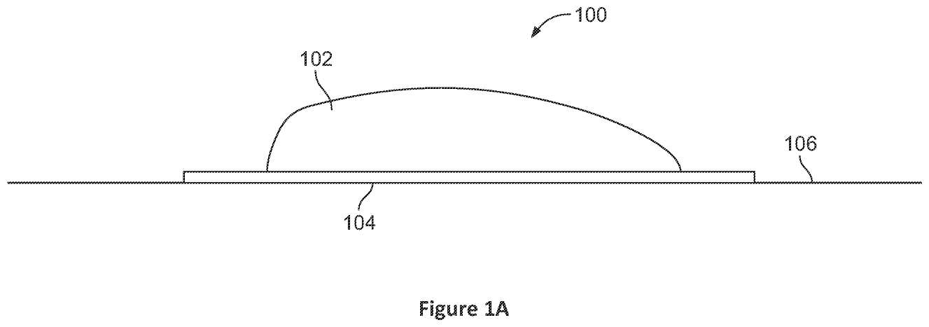

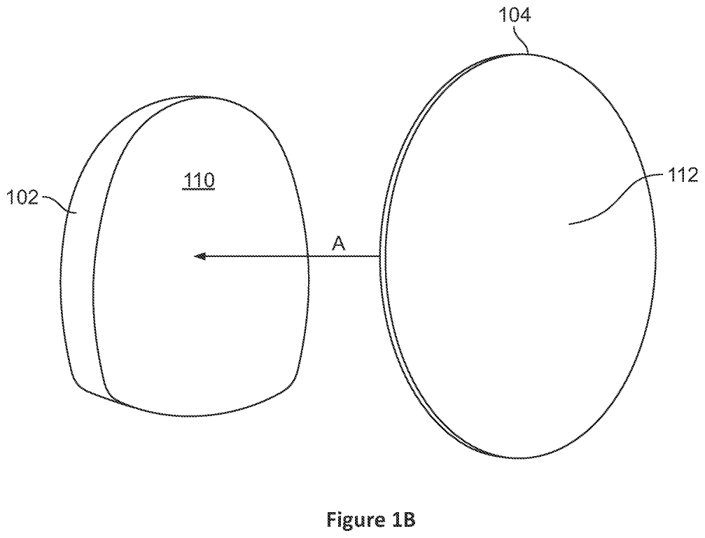

FIG. 1A depicts an illustrative on-body medical device 100 for an exemplary embodiment. The on-body medical device 100 may be, for example, a monitor or a sensor. For example, the on-body medical device 100 may be a heart rate monitor, a blood pressure monitor, a glucose sensor, a ketone sensor, or the like. The on-body medical device 100 may be a medicament delivery device. The medicament delivered by the on-body medical device 100 may be insulin, pramlintide, GLP-1 or a co-formulation of two or more of the foregoing for lowering a user's glucose level, or may be glucagon for raising a user's glucose level. The medicament may be a glucagon-like peptide (GLP)-1 receptor agonist for lowering glucose or slowing gastric emptying, thereby delaying spikes in glucose after a meal. Alternatively, the medicament delivered by the medicament delivery device 102 may be one of a pain relief agent, a chemotherapy agent, an antibiotic, a blood thinning agent, a hormone, a blood pressure lowering agent, an antidepressant, an antipsychotic, a statin, an anticoagulant, an anticonvulsant, an antihistamine, an anti-inflammatory, a steroid, an immunosuppressive agent, an antianxiety agent, an antiviral agent, a nutritional supplement, a vitamin, or co-formulations of two or more of the above.

More generally, the on-body medical device 100 is a device that is adhered to the user and serves a medical purpose. The on-body medical device 100 includes at least one housing 102 for housing components of the on-body medical device. The at least one housing may be positioned on or in a tray, which may previously have been attached to the skin of the user, and the tray may be attached to the adhesive layer. Alternatively, the at least one housing 102 may be attached to the adhesive layer as described in exemplary embodiments below. The at least one housing 102, may be made of a material, such as a plastic, a composite, a metal or a combination thereof. For example, suppose that the on-body medical device 100 is an insulin pump. In that case, the at least one housing 102 may house the mechanical and electrical components of the insulin pump, like an insulin reservoir, a microprocessor, a memory, a pump, batteries and the like. An adhesive layer 104 is secured to a surface of the on-body medical device 100. In FIG. 1A, the adhesive layer 104 is secured to a bottom exterior surface of the housing by thermal weld or another securing mechanism, such as an adhesive or a mechanical fastener. In other embodiments, the adhesive layer 104 is secured to a bottom exterior surface of a tray by thermal weld or another securing mechanism, such as an adhesive or mechanical fastener, and the tray may serve as a foundation for the at least one housing 102 and the various other elements of the on-body medical device 100. The adhesive layer 104 includes multiple strengths of adhesives on it for securing the on-body medical device 100 to the skin 106 of a user. The adhesive layer 104 may be formed of a base or substrate material upon which the adhesives are coated, applied, or secured.

FIG. 1B depicts a partially exploded view of a bottom exterior surface 110 of the housing 102 to which the adhesive layer 104 is secured as indicated by arrow A. The circumference of the outer border of the adhesive layer 104 may be larger than the circumference of the outer border of the surface 110. Surface 112, also referred to as first surface, of the adhesive layer 104 is the surface on which the adhesives are present and is the surface that is secured to the skin 106 of the user (when the on-body medical device 100 is attached to the user).

In some embodiments, the surface to which the adhesive layer 104 is secured is not part of the housing 102. Instead the surface may be part of a stand, feet or other element that is configured to be skin-facing (i.e., oriented to face the skin of the user). Accordingly, in some embodiments, the surface is an outer surface of the on-body medical device 100. That the surface is skin-facing does not require the surface to be in direct contact with the skin of the user, when the medical device 100 is attached to the user, in particular the adhesive layer 104 may be disposed between the surface and the skin 106 of the user. Moreover, there need not be a single adhesive layer. There may be multiple adhesive layers for multiple contact areas with the skin of the user.

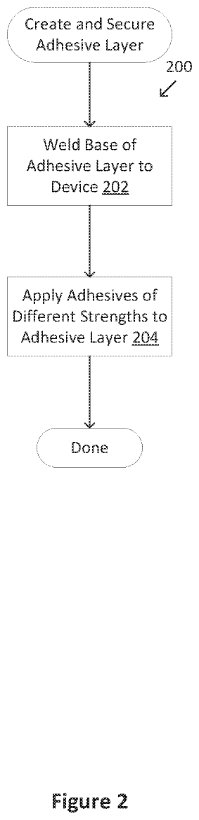

FIG. 2 depicts a flowchart 200 of illustrative steps that may be performed in exemplary embodiments to create and secure the adhesive layer. The base or substrate (hereinafter “base”) of the adhesive layer may be a layer of a material, such as a layer of a polyester woven material. At 202, this base of the adhesive layer is welded to a skin-facing surface of the on-body medical device. The skin-facing surface of the on-body medical device may be the first surface described above. The weld may be a thermal weld formed by melting a plastic weld structure formed on the surface. As was mentioned above, in other embodiments, the base may be secured to the surface by other means, like an adhesive or a mechanical fastener. At 204, adhesives of different strengths may be applied to the base of the adhesive layer. This application may entail coating portions of the adhesive layer with the adhesives. In some embodiments, the bottom layer of adhesive on the adhesive layer may be of greater strength and an adhesive of lower strength may be applied on top of the greater strength adhesive at selected areas. Those skilled in the art will appreciate that various techniques may be used to apply the adhesives to the adhesive layer.

Strength of the adhesives may be measured and classified in different ways. For example, the adhesives may be measured by peel strength. The peel strength may be determined for example according to ISO 8510-2:2006-12 or DIN EN 28510-1:2014-07. The peel strength may be expressed as N/mm. The adhesives strengths also may be measured and classified by how long the adhesive are intended to be adhered to the skin of a typical user experiencing typical use conditions. For example, an adhesive may be classified as a two day wear time adhesive if the adhesive is intended to securely hold for two days on the skin of a typical user with average skin properties (e.g., properties such as perspiration level at the particular skin surfaces where on-body medical devices are typically worn).

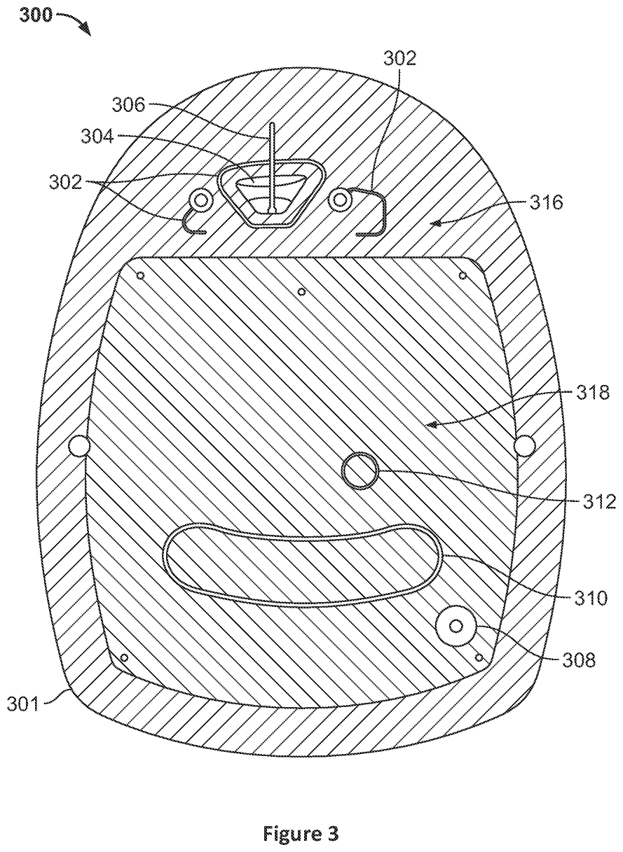

FIG. 3 depicts the bottom surface of an on-body medical device 300 with an adhesive layer 301 attached. In this depicted example, the on-body medical device 300 is an insulin pump that delivers insulin to a user. The adhesive layer 301 includes adhesives of two different strengths. A stronger adhesive 316 is on the outer perimeter of the adhesive layer and around a needle/cannula assembly that includes an opening 304 through which a needle and/or cannula 306 may exit to puncture the skin of the user and through which the needle or cannula may retract back into the housing of the on-body medical device. Thermal welds 302 are formed around the needle/cannula assembly to ensure that the adhesive layer 301 does not detach from the bottom surface of the housing. The higher strength adhesive 316 is positioned around the needle/cannula assembly to ensure that the needle and/or cannula 306 does not move or shift once the adhesive layer 301 is applied to the skin of the user. A weaker adhesive 318 is on the adhesive layer 301 in the interior area inside the perimeter of the adhesive layer 301. The weaker adhesive 318 covers the area over the large weld 310 and valve 312. The weaker adhesive surrounds a needle port 308 for adding insulin to a reservoir.

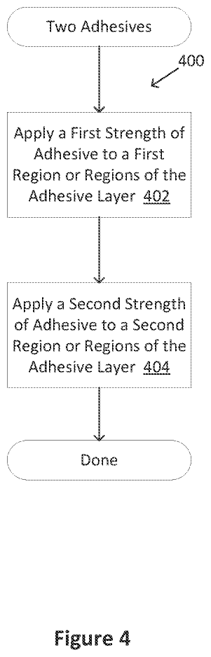

FIG. 4 depicts a flowchart 400 of illustrative steps that may be performed in exemplary embodiments to create an adhesive layer with two strengths of adhesive like that shown in FIG. 3. At 402, an adhesive of a first strength is applied to a first region or regions of the adhesive layer. For example, in FIG. 3, the stronger adhesive 316 may be applied to the entire adhesive layer 301 or merely to the perimeter and around the needle/cannula assembly. At 404, an adhesive of a second strength is applied to a second region or regions of the adhesive layer. With respect to the example of FIG. 3, the weaker adhesive 318 is applied to cover the indicated interior region either by direct application of the weaker adhesive 318 to the base of the adhesive layer 301 or on top of the stronger adhesive in the indicated interior region.

The stronger adhesive 316 may be 10% to 100% stronger than the weaker adhesive, or more particularly, 10% to 50%; or in an exemplary embodiment, 20% stronger than the weaker adhesive 318. The strength may be measured as peel strength or as wear time. “Wear time” may refer to an average wear time for a typical user (with typical skin properties) under typical use conditions. The peel strength of the stronger adhesive may be at least 20% stronger than the peel strength of the weaker adhesive 318 in an exemplary embodiment. Alternatively, the wear time of the stronger adhesive 316 may be at least 20% greater than the wear time of the weaker adhesive 318 in an exemplary embodiment. For example, the weaker adhesive may be a 1 or 2 day “wear time” adhesive, whereas the stronger adhesive may be a 3 or 4 day “wear time” adhesive, for an on-body medical device intended to be adhered to the user for 3 or 4 days. Thus, the stronger adhesive may be 50% or even 100% stronger in some instances, as suggested above.

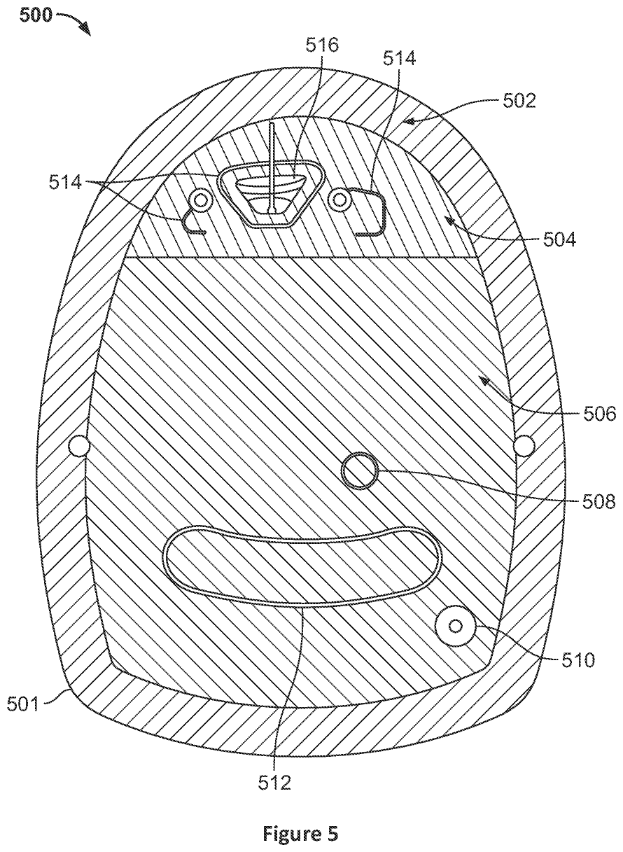

As was mentioned above, the adhesive layer also may have more than two strengths of adhesives on it. FIG. 5 depicts an illustrative bottom surface of an on-body medical device 500 with an adhesive layer 501 having adhesives 502, 504 and 506 of different strengths. A stronger adhesive 502 is positioned around the perimeter of the adhesive layer 501. An intermediate strength adhesive 504 is positioned around the needle/cannula hole 516 and welds 514 to provide enhanced adhesive strength relative to the weaker adhesive 506. The weaker adhesive 506 is positioned around the interior of the adhesive layer around the region where valve 508, port 510 and weld 512 are located. This approach provides enhanced strength around the needle/cannula assembly but with an adhesive that is not as strong as the stronger adhesive. As such, the portion with the intermediate strength adhesive 514 is less painful to peel off and less likely to cause skin damage when peeled off than the same portion of the adhesive layer in the embodiment of FIG. 3. The stronger adhesive 502 may be 10% to 50% stronger than the intermediate strength adhesive 504, or more particularly, 10% to 30%; or in an exemplary embodiment, approximately 15% stronger than the intermediate strength adhesive 504. Similarly, the intermediate strength adhesive 504 may be 10% to 50% stronger than the weaker adhesive 506, or more particularly, 10% to 30%; or in an exemplary embodiment, approximately 15% stronger than the weaker strength adhesive 506.

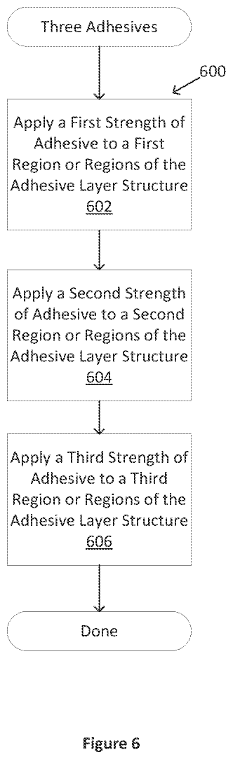

FIG. 6 depicts a flowchart 600 of illustrative steps that may be performed in exemplary embodiments to create an adhesive layer with three strengths of adhesives. At 602, a first strength of adhesive may be applied to a first region or regions of the adhesive layer as part of an adhesive layer. For example, in FIG. 5, the stronger adhesive 502 is applied to the perimeter of the adhesive layer 501. At 604, a second strength of adhesive may be applied to a second region or regions of the adhesive layer. For example, in FIG. 5, the intermediate strength adhesive 504 is applied inside the perimeter near the needle/cannula assembly or patient interface, or portion where an element extends out of the at least one housing and/or tray and through the skin of the user. At 606, a third strength of adhesive may be applied to a third region or regions of the adhesive layer as part of an adhesive layer. In FIG. 5, the third region is the region where the weaker adhesive 506 is applied.

It should be appreciated that adhesives with more than three strengths may be applied to an adhesive layer. The number of adhesive strengths used on a particular adhesive layer may depend on the needs for a variety of adhesion strengths on the adhesive layer.

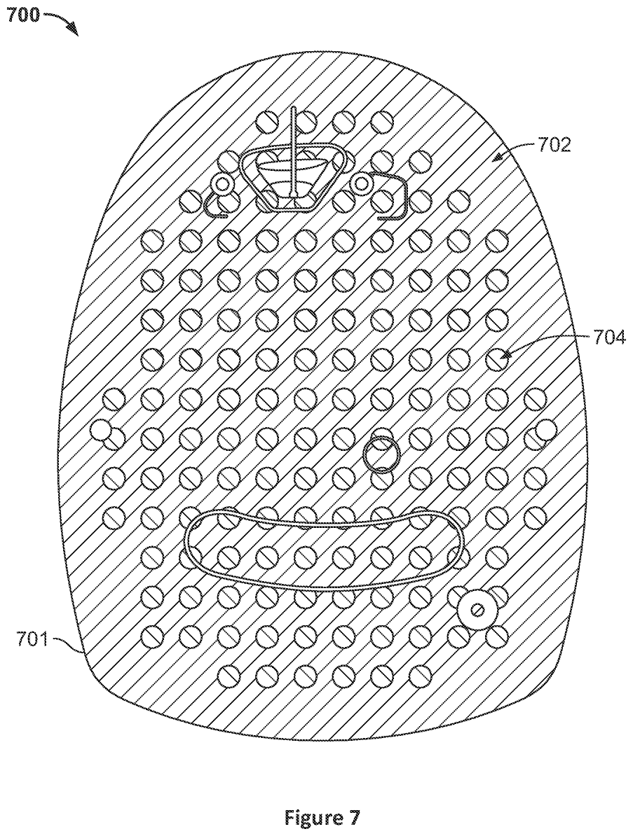

The adhesive layer need not be limited to having adhesives of different strengths applied to large continuous regions. Instead, an adhesive of a given strength may be applied to non-continuous discrete locations on the adhesive layer. FIG. 7 depicts the bottom surface 700 of an on-body medical device with an adhesive layer 701. The adhesive layer 701 has a grid of discrete points or islands (both are referred to as “islands” herein) 704 of the weaker strength adhesive. The islands 704 are surrounded, in particular the islands' 704 entire perimeter is surrounded, by a region 702 to which the stronger adhesive is applied. The region 702 encompasses the perimeter of the adhesive layer 701 and the spaces between the islands 704. This pattern of adhesives provides secure support around the perimeter of the adhesive layer 701 and adds some additional strength in the interior of the adhesive layer 701.

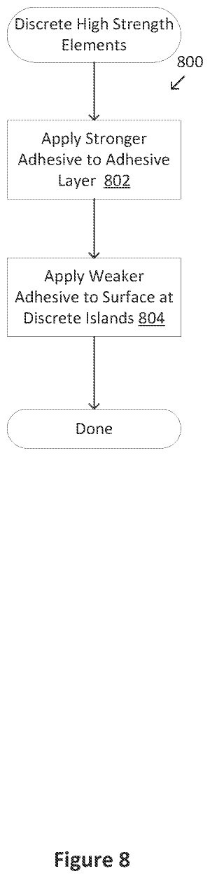

FIG. 8 depicts a flowchart 800 of illustrative steps that may be performed in exemplary embodiments to create an adhesive layer with islands of adhesive like that shown in FIG. 7. At 802, a stronger adhesive is applied to the adhesive layer. The stronger adhesive may be applied across the entire surface of the adhesive layer where there are no holes, ports, or the like. Alternatively, the stronger adhesive may be applied with a mask or other construct to leave the islands 704 without adhesive. At 804, the weaker adhesive is applied on top of the stronger adhesive at the islands 704 using a mask or other construct. It should be noted that in case the weaker adhesive is applied on to top of the stronger adhesive, the islands may be defined by their entire perimeter being surrounded by a stronger adhesive at the interface of the weaker adhesive and the stronger adhesive.

Alternatively, the islands 704 instead may be where the stronger adhesive is applied, and the area 702 may be where the weaker adhesive is applied or even no adhesive is applied. Moreover, in some embodiments, the islands 704 may be regions where no adhesive is applied and an intermediate strength adhesive or stronger strength adhesive is applied to region 702.

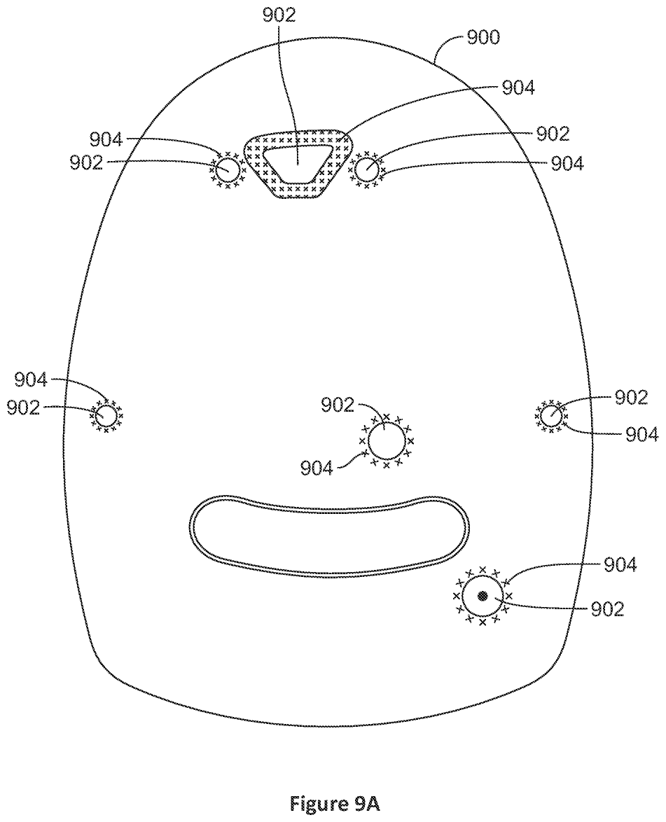

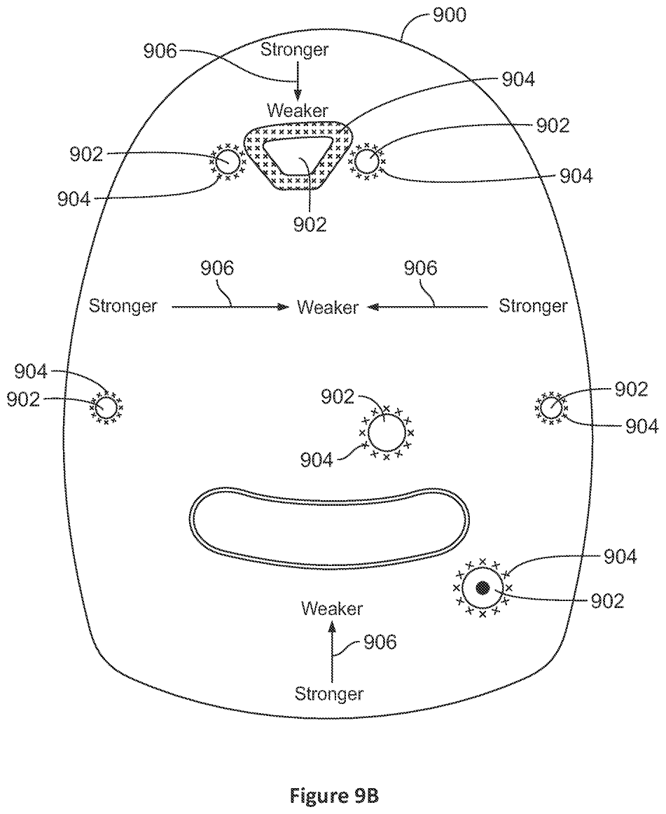

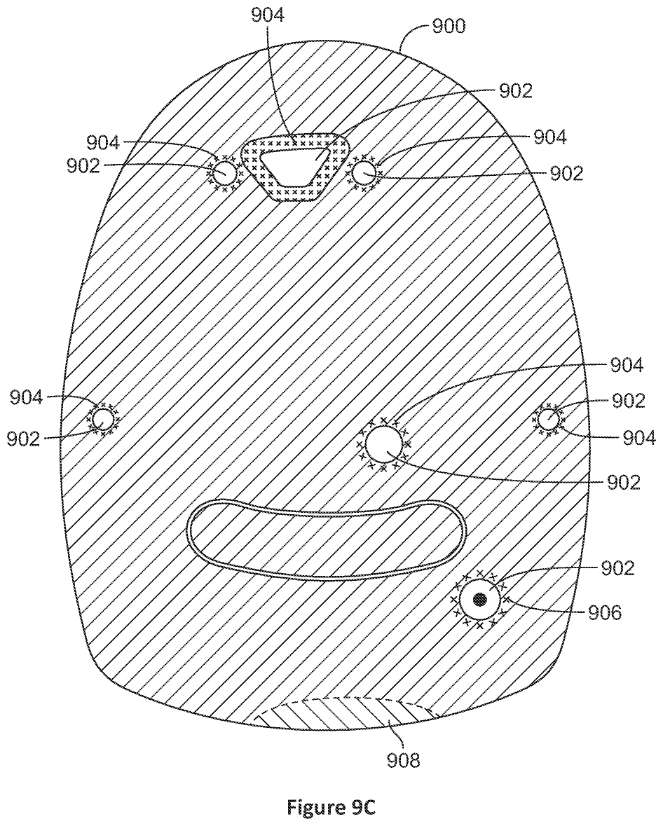

It may be desirable to put stronger adhesive around holes or other structures that protrude through the adhesive layer to prevent peeling of the adhesive layer at edges that surround such holes or structures. FIG. 9A depicts the adhesive layer 900 with examples of holes and structures 902 around which stronger adhesive may be applied. These holes or structures 902 may be areas where an air valve, a fill hole, a needle or cannula hole, a patient interface hole, a speaker opening, a light or LED opening, a reset button, a kill switch, or other element is located and necessitates a hole or space or break in the adhesive layer. These holes or structures 902 may also represent areas where a manufacturing operation necessitates holes, for example, to hold the adhesive layer 900 during a step in the manufacturing process. In an exemplary embodiment, it may be desirable to form a stronger adhesive 904 around all or most of the holes and/or structures 902 in the adhesive layer 900 so as to better secure the adhesive layer 900 to the skin at these locations. Because the adhesive layer 900 typically begins to pull apart from the skin surface at its edges, including potentially at edges of holes in the adhesive layer 900, using a stronger adhesive at these locations would enhance the wear time of the adhesive and medical device to the user's skin, while not substantially increasing the discomfort on the user's skin as may happen if the entire surface of the adhesive layer 900 were formed with a stronger adhesive. Examples of a “stronger” adhesive are described above in terms of % of peel strength and/or wear time. A less strong adhesive may be used elsewhere for the adhesive layer as indicated by the cross hatching on the adhesive layer 900 where the stronger adhesive is not applied in FIG. 9A.

In another exemplary embodiment, the adhesive may form a gradient of strength from an outer perimeter of the adhesive layer 900 where the adhesive may be stronger, and gradually weaken moving toward the center of the adhesive layer 900 where the adhesive typically doesn't need to be as strong since the interior portions may not experience the same stresses as the perimeter of the adhesive layer 900. An example of such a gradient of strength on the adhesive layer 900 is shown in FIG. 9B. The stronger adhesive at the outer perimeter may be 10% to 100% stronger than the weaker adhesive at the center of the adhesive layer 900, or more particularly, 10% to 50%; or in an exemplary embodiment, 20% stronger. The adhesive may gradually weaken at positions on the adhesive layer 900 located radially inward from the perimeter until it becomes what has been referred to above as the “weaker adhesive.” Arrows 906 indicate the gradient from stronger adhesive on the perimeter to weaker adhesive in the radial inner positions that are closer to the center of the adhesive layer 900. This gradient may be created by applying different amounts or different types of adhesive across the surface of the adhesive layer 900, such that the perimeter has a greater amount or (stronger) type of adhesive material, in particular a greater amount of a stronger type of adhesive, than the interior or near the center of the adhesive layer 900, which may result in a stronger adhesive property.

In a further exemplary embodiment as depicted in FIG. 9C, a portion of the perimeter of the adhesive layer 900 may have a weaker adhesive then other portions of the perimeter or than other portions of the adhesive layer 900. For example, one portion (such as a portion 908 of the bottom end) of the adhesive layer 900 may have a weaker adhesive (or in some embodiments, no adhesive) thereby making it easier to separate from a user's skin, so as to allow the user to insert their finger at that portion 908 under the adhesive layer and above the skin when it comes time to remove the adhesive layer 900 from the skin (e.g., at the end of life of the on-body medical device). This portion 908 may be arcuate in shape or in the shape of a fingertip. By allowing a user to insert their fingertip at this weaker adhesive portion 908 when it comes time to remove the adhesive layer 900 from the skin, it may be easier for the user to start the adhesive removal process of the rest of the adhesive layer 900. Once the user is able to separate or lift this portion of the adhesive layer 900, the user can grab this separated portion and gradually lift the adhesive layer 900 in a peeling fashion to remove the remainder of the adhesive layer 900, where the remainder of the adhesive layer 900 has a stronger adhesive than this particular portion of the perimeter.

While exemplary embodiments have been described herein, various changes in form and detail may be made without departing from the intended scope of the appended claims.

1. An on-body medical device comprising:
a skin-facing surface configured for facing skin of a user; and
an adhesive layer secured to the skin-facing surface for securing the on-body medical device to the skin of the user, comprising:
a stronger adhesive positioned around an outer perimeter of the adhesive layer, and
a weaker adhesive positioned inside the outer perimeter of the adhesive layer,
wherein the stronger adhesive is at least 20% stronger than the weaker adhesive.

2. The on-body medical device of claim 1, further comprising:
a needle and/or cannula insertion assembly for inserting a needle and/or cannula through the skin of the user to deliver a medicament to the user,
an opening in the skin-facing surface, and
an opening in the adhesive layer that is aligned with the opening in the skin facing surface to create a path for the needle and/or cannula to pass for enabling the needle and/or the cannula to be inserted through the skin of the user.

3. The on-body medical device of claim 2, wherein the stronger adhesive also surrounds a perimeter of the opening in the adhesive layer.

4. The on-body medical device of claim 2, further comprising an intermediate strength adhesive that surrounds the opening in the adhesive layer, wherein the intermediate strength adhesive is weaker than the stronger adhesive but stronger than the weaker adhesive.

5. The on-body medical device of claim 1, wherein the weaker adhesive is a 1 day wear time adhesive or a 2 day wear time adhesive.

6. The on-body medical device of claim 5, wherein the stronger adhesive is a 3 day wear time adhesive or a 4 day wear time adhesive.

7. The on-body medical device of claim 1, wherein the stronger adhesive is a 3 day wear time adhesive or a 4 day wear time adhesive.

8. The on-body medical device of claim 1, wherein the on-body medical device is a medicament delivery device.

9. The on-body medical device of claim 8, wherein the medicament delivery device is an insulin pump.

10. The on-body medical device of claim 9, wherein the skin-facing surface is a surface on the housing of the on-body medical device.

11. The on-body medical device of claim 1, further comprising:
a hole in the adhesive layer; and
the stronger adhesive positioned on the adhesive layer around a perimeter of the hole.

12. An on-body medical device, comprising:
a housing having a skin-facing surface configured for facing the skin of a user; and
an adhesive layer secured to the skin-facing surface of the housing for securing the on-body medical device to the skin of the user, comprising:
islands on the adhesive layer where no adhesive is present or a weaker strength adhesive is present, and
a stronger strength adhesive applied around a perimeter of the adhesive layer and also applied around a region interior to the perimeter so as to surround the islands with the stronger strength adhesive, wherein the stronger strength adhesive is at least 20% stronger than the weaker strength adhesive.

13. The on-body medical device of claim 12, wherein the islands are periodically spaced apart.

14. The on-body medical device of claim 13, wherein the islands are arranged in a grid.

15. The on-body medical device of claim 12, wherein the islands have a weaker adhesive present and the weaker adhesive is a 1 day wear time or a 2 day wear time adhesive.

16. The on-body medical device of claim 12, wherein the stronger adhesive is a 3 day wear term or a 4 day wear term adhesive.

17. An on-body medical device, comprising:
a skin-facing surface configured for facing the skin of a user; and
an adhesive layer secured to the skin-facing surface for securing the on-body medical device to the skin of the user, comprising:
islands on the adhesive layer where a stronger strength adhesive is present, and
a weaker strength adhesive applied around a perimeter of the adhesive layer and also applied around a region interior to the perimeter so as to surround the islands with the weaker strength adhesive, wherein the stronger strength adhesive is at least 20% stronger than the weaker strength adhesive.

18. The on-body medical device of claim 17, wherein the islands are periodically spaced apart.

19. The on-body medical device of claim 17, wherein the islands are arranged in a grid.

20. The on-body medical device of claim 17, further comprising:
a needle and/or cannula insertion assembly for inserting a needle and/or cannula through the skin of the user to deliver a medicament to the user;
an opening in the skin-facing surface; and
a corresponding opening in the adhesive layer for enabling the needle and/or the cannula to be inserted through the skin of the user.
